SEQUENCE LISTING (11 sequences)

SEQ ID NO: 1 - DNA, 348 bp, Arabidopsis thaliana
atggctttcgctttgaggttcttcacatgccttgttttaacggtgtgcatagttgcatcagtcgatgctgcaatctcatgtggcacagtggcaggtagcttggctccatgtgcaacctatctatcaaaaggtgggttggtgccaccttcatgttgtgcaggagtcaaaactttgaacagtatggctaaaaccacaccagaccgccaacaagcttgcagatgcatccagtccactgcgaagagcatttctggtctcaacccaagtctagcctctggccttcctggaaagtgcggtgttagcattccatatccaatctccatgagcactaactgcaacaacatcaagtga

SEQ ID NO: 2 - protein, 115 aa, Arabidopsis thaliana
MAFALRFFTCLVLTVCIVASVDAAISCGTVAGSLAPCATYLSKGGLVPPSCCAGVKTLNSMAKTTPDRQQACRCIQSTAKSISGLNPSLASGLPGKCGVSIPYPISMSTNCNNIK

SEQ ID NO: 3 - DNA, 393 bp, artificial
  LTP fusion protein DNA
atgcaccatcatcatcatcattcttctggtctggtgccacgcggttctggtatgaaagaaaccgctgctgctaaattcgaacgccagcacatggacagcccagatctgggtaccgcaatctcatgtggcacagtggcaggtagcttggctccatgtgcaacctatctatcaaaaggtgggttggtgccaccttcatgttgtgcaggagtcaaaactttgaacagtatggctaaaaccacaccagaccgccaacaagcttgcagatgcatccagtccactgcgaagagcatttctggtctcaacccaagtctagcctctggccttcctggaaagtgcggtgttagcattccatatccaatctccatgagcactaactgcaacaacatcaagtga

SEQ ID NO: 4 - protein, 130 aa, artificial
  LTP fusion protein
MHHHHHHSSGLVPRGSGMKETAAAKFERQHMDSPDLGTAISCGTVAGSLAPCATYLSKGGLVPPSCCAGVKTLNSMAKTTPDRQQACRCIQSTAKSISGLNPSLASGLPGKCGVSIPYPISMSTNCNNIK

SEQ ID NO: 5 - DNA, 667 bp, Thellungiella halophila
aattcggcacgaggcctcgtgccgaattcggcacgaggcaaaaaacaaccttagaaaacaaaagtcaactaaatctccctattttcttatcacaaaaaaggtaaagcaaaacacaatggctttggctctgaggttcttcacatgccttgttttgacggtgtgcatagttgcatcagtagatgcagcaatctcatgtggcacagtggcaagcagcttggctccatgtgcaggctacctaacaaaaggaggcgcggtgcccgctccgtgctgtgctggagtgtcaaagttgaacggcatggctaaaaccacaccagaccgccaacaagcttgcaaatgcctaaaggccgcagcacagagcatcaacccaagtctagcctctggccttcctggaaagtgcggtgttagcattccctatcccatctccatgagcaccaactgcgacaacgtcaagtgaattgagcactcacatcgtgtggatgaagatgcatggtttagcataagtaaaataaaaacgtctgtgtacgctgatctcagcttgccctagtttatcttgttttattttggttgttgaaagtttgtcatcttactttgtaatcttttgcttttattatattgtattatggttaaagtatgatagtagtaccttcttaaaaaaaaaaaaaaaaaa

SEQ ID NO: 6 - protein, 112 aa, Thellungiella halophila
MALALRFFTCLVLTVCIVASVDAAISCGTVASSLAPCAGYLTKGGAVPAPCCAGVSKLNGMAKTTPDRQQACKCLKAAAQSINPSLASGLPGKCGVSIPYPISMSTNCDNVK

SEQ ID NO: 7 - DNA, 586 bp, Brassica napus
gacaaaagagaaaagcaaaagacaatggcttcggctctgagttttttcacatgccttgttttgactgtgtgcatagttgcatcagtagatgcagcaatctcatgtggcacagtgacaagtaacttggctccatgtgccgtctatctaatgaaaggcgggccggtgccagctccatgctgcgccggagtttcaaaattgaacagtatggctaaaaccacaccggaccgccaacaagcatgtaaatgcctaaagaccgctgcaaagaacgtcaatccaagtctagcctctagccttcctggaaagtgcggtgttagcattccctatcccatctccatgagcactaactgcgacaccgtcaagtgaaatgggaactcatatatcatcgtgaggatgaacagtatggtctagcataaataaaagagtgtctatctacactgacctcagcatgtcctagtttgtcttgtttttattatagtcgaaagtttgtcatgttactttgtaatcttttgctttatattgtgtcttaatgatgtttaagatatgataatattatcctattaaaaaaaaaaaaaaaaaaaaaaaaaaa

SEQ ID NO: 8 - protein, 112 aa, Brassica napus
MASALSFFTCLVLTVCIVASVDAAISCGTVTSNLAPCAVYLMKGGPVPAPCCAGVSKLNSMAKTTPDRQQACKCLKTAAKNVNPSLASSLPGKCGVSIPYPISMSTNCDTVK

SEQ ID NO: 9 - DNA, 30 bp, artificial
  PCR primer
aattgcggccgctcacttgatgttgttgca

SEQ ID NO: 10 - DNA, 27 bp, artificial
  PCR primer
attggtaccgcaatctcatgtggcaca

SEQ ID NO: 11 - DNA, 30 bp, artificial; identical to SEQ ID NO: 9
  PCR primer

(19) United States
(12) Patent Application Publication
(10) Pub. No.: US 2004/0191851 A1
(43) Pub. Date: Sep. 30, 2004

(54) Methods for the identification of inhibitors of lipid transfer protein activity in plants

(76) Inventors: Angelika Reichert, Durham, NC (US); Lining Guo, Chapel Hill, NC (US); Keith Davis, Durham, NC (US); Adel Zayed, Durham, NC (US); Robert Ascenzi, Cary, NC (US); Douglas Boyes, Chapel Hill, NC (US); Rao Mulpuri, Apex, NC (US); Neil Hoffman, Chapel Hill, NC (US); Susanne Kjemtrup, Chapel Hill, NC (US); Carol Hamilton, Apex, NC (US); Jeffrey Woessner, Hillsborough, NC (US); Jorn Gorlach, Manchester, NJ (US)

Correspondence Address: PARADIGM GENETICS, INC, 108 ALEXANDER DRIVE, P O BOX 14528, RTP, NC, 27709-4528, US

(21) Appl. No.: 10/401,049

(22) Filed: Mar. 27, 2003

(51) Int. Cl. C12Q 1/68; C12Q 1/42; A01N 63/00; C12N 9/16

(52) U.S. Cl. 435/21; 435/196; 504/117

(57) ABSTRACT

The present inventors have discovered that Lipid Transfer Protein (LTP) is essential for plant growth. Specifically, the inhibition of LTP gene expression in plant seedlings results in reduced growth and chlorosis. Thus, LTP is useful as a target for the identification of herbicides. Accordingly, the present invention provides methods for the identification of herbicides by measuring the activity of an LTP in the presence and absence of a compound, wherein an alteration of LTP activity in the presence of the compound indicates the compound as a candidate for a herbicide.

### FIELD OF THE INVENTION

The invention relates generally to plant molecular biology. In particular, the invention relates to methods for the identification of herbicides.

### BACKGROUND OF THE INVENTION

Lipid Transfer Proteins (LTP's) are characterized by their ability to transfer phospholipids between membranes and to bind fatty acids or lysoderivates (Kader J. C. (1997) Trends Plant Science 2:66-70; Zachowski et al. (1998) Eur. J. Biochem. 257:443-448). LTP's transfer phosphatidylcholin (PC), phosphatidylinositol (PI), and phosphatidylethanolamine (PE) between various membranes. Galactolipids, but not triacylglycerols, are also transferred by LTP's (Kader (1997), supra).

LTP's are a family of small, soluble proteins with a basic character. LTP's are found in abundance in higher plants and are thought to be involved in a variety of functions including membrane formation, regulation of intracellular fatty acid pools, participation in cutin formation, embryogenesis, defense reactions, symbiosis, and environmental adaptation (Kader (1997), supra). Two main groups of LTPs, LTP1 and LTP2, have been identified with molecular masses of about 9 and 7 kDa., respectively (Charvolin et al. (1999) Eur. J. Biochem. 264:562-568). The crystallographic and solution structures of several LTP's have been solved (Charvolin et al. (1999), supra; Han et al. (2001) J. Mol. Biol. 308:263; Tassin-Moindrot et al. (2000) Eur. J. Biochem. 267:1117-1124; Poznanski et al. (1999) Eur. J. Biochem. 259:692-708). The crystallographic structures of several LTP's complexed with lipid substrates show that the hydrophobic tail of the lipid substrate is inserted into an internal hydrophobic cavity running through the molecule (Kader (1997), supra; Poznanski et al. (1999), supra). Lipid substrate binding to LTP's has been studied by several groups (Zachowski et al. (1998), supra; Tassin-Moindrot et al. (2000), supra; Grosbois et al. (1993) Biochem. Biophys. Acta. 1170:197-203; Douliez et al. (2001) Eur. J. Biochem. 268:1400-1403). Zachowski et al. (1998) found fatty acids with medium chain length, between 16 and 19 carbons, to display the highest affinity to maize LTP.

LTP1 are synthesized with an N-terminal signal peptide, consistent with the location of these proteins in extracellular layers, i.e. cell walls or cutin, and in vacuolar structures (Charvolin et al. (1999), supra). In Arabidopsis thaliana, LTP1 has been found to be located in the cell wall (Kader J. C. (1996) Annu. Rev. Plant Physiol. Plant Mol. Biol. 47:627-654). It has been suggested that the in vivo function of LTP's is secretion and/or deposition of extracellular lipophilic material, including cutin.

The present invention discloses LTP as a target for the evaluation of plant growth regulators, especially herbicide compounds, in plants.

### SUMMARY OF THE INVENTION

The present inventors have discovered that antisense expression of a LTP cDNA in Arabidopsis causes chlorosis and stunted growth. Thus, the present inventors have discovered that LTP is essential for normal plant development and growth, and is useful as a target for the identification of herbicides. Accordingly, in one embodiment the present invention provides methods for the identification of compounds that inhibit LTP activity, comprising: contacting a compound with a LTP and detecting the presence or absence of binding between the compound and the LTP, wherein binding between the compound and the LTP indicates the compound as a herbicide target. In another embodiment of the invention, methods are provided for the identification of compounds that inhibit LTP enzyme activity, comprising: contacting a LTP polypeptide with a lipid substrate in the presence and absence of a compound; and determining a change in binding of the lipid substrate in the presence and absence of the compound, wherein a change in binding for the lipid substrate indicates that the compound is a candidate herbicide.

### DETAILED DESCRIPTION OF THE INVENTION

Definitions

The term “bDNA” refers to branched DNA.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions mediates the binding of two molecules to each other.

As used herein, the term “cDNA” means complementary deoxyribonucleic acid.

As used herein, the term “dI” means deionized.

As used herein, the term “ELISA” means enzyme-linked immunosorbent assay.

As used herein, the term “GUS” means β-glucouronidase.

The term “herbicide”, as used herein, refers to a compound useful for killing or suppressing the growth of at least one plant, plant cell, plant tissue or seed.

As used herein, the term “homologous LTP” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 40-100% in ascending order to Arabidopsis LTP protein (SEQ ID NO:2) and at least 10%, 25%, 50%, 75%, 80%, 90%, 95%, or 99% activity or each integer unit of activity from 10-100% in ascending order of the activity of Arabidopsis LTP protein (SEQ ID NO:2). Examples of homologous LTP's include, but are not limited to, LTP from Thellungiella halophia and LTP from Brassica napus.

As used herein, the term “HPLC” means high pressure liquid chromatography.

The term “inhibitor,” as used herein, refers to a chemical substance that inactivates the enzymatic activity of LTP or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide is “introduced” into a plant cell by any means, including transfection, transformation or transduction, electroporation, particle bombardment, agroinfection and the like. The introduced polynucleotide is maintained in the cell stably if it is incorporated into a non-chromosomal autonomous replicon or integrated into the plant chromosome. Alternatively, the introduced polynucleotide is present on an extra-chromosomal non-replicating vector and be transiently expressed or transiently active.

As used herein, the term “LB” means Luria-Bertani media.

As used herein, the terms “Lipid Transfer Protein (LTP)” and “Lipid Transfer Protein (LTP) polypeptide” refer to an enzyme that catalyzes the transfer of lipid substrates. Lipid substrates include phospholipids, fatty acids, galactolipids and lysoderivates. Examples of particular lipid substrates include, but are not limited to, phosphatidylcholine (PC), phosphatidylinositol (PI), phosphatidylethanolamine (PE), 1-pyrenedodecanoic acid (PDA), stearic acid, ω-hydroxypalmitic acid, myristoylglycero-phosphatidylcholine, prostaglandin B2, and lauric acid.

As used herein, the term “Ni-NTA” refers to nickel sepharose.

As used herein, the term “PCR” means polymerase chain reaction.

The “percent (%) sequence identity” between two polynucleotide or two polypeptide sequences is determined according to the either the BLAST program (Basic Local Alignment Search Tool, Altschul and Gish (1996) Meth Enzymol 266: 460-480; Altschul (1990) J Mol Biol 215: 403-410) or using Smith Waterman Alignment (Smith and Waterman (1981) Adv Appl Math 2:482) using the default settings and the version current at the time of filing). It is understood that for the purposes of determining sequence identity when comparing a DNA sequence to an RNA sequence, a thymine nucleotide is equivalent to a uracil nucleotide.

As used herein, the term “PGI” means plant growth inhibition.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant LTP” is meant a protein found in at least one plant, and which catalyzes the transfer of lipid substrates. The LTP is from any plant, including monocots, dicots, C3 plants, C4 plants, and/or plants that are neither C3 nor C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

As used herein, the term “SDS-PAGE” means sodium dodecyl sulfate-polyacrylimide gel electrophoresis.

The term “specific binding” refers to an interaction between LTP and a molecule or compound, wherein the interaction is dependent upon the primary amino acid sequence or the conformation of LTP.

The present inventors have discovered that inhibition of LTP gene expression inhibits the growth and development of plant seedlings. Thus, the inventors are the first to demonstrate that LTP is a useful target for the identification of herbicides.

Accordingly, the invention provides methods for identifying compounds that inhibit LTP protein activity. Such methods include ligand binding assays, assays for enzyme activity and assays for LTP gene expression. The compounds identified by the methods of the invention are useful as herbicides.

Thus, in one embodiment, the invention provides a method for identifying a compound as a candidate for a herbicide, comprising: contacting a LTP with a compound; and detecting the presence and/or absence of binding between the compound and the LTP, wherein binding indicates that the compound is a candidate for a herbicide.

By “LTP” is meant an enzyme that catalyzes the transfer of lipid substrates. In one embodiment of the invention, the LTP has the amino acid sequence of a naturally occurring LTP found in a plant, animal or microorganism. In another embodiment of the invention, the LTP has an amino acid sequence derived from a naturally occurring sequence. In another embodiment the LTP is a plant LTP. Homologous LTP's are useful in another embodiment of the invention.

One example of a cDNA encoding an Arabidopsis LTP is set forth in SEQ ID NO:1 (TIGR database locus At5g59320/mnc17). The LTP polypeptide encoded by SEQ ID NO:1 is set forth in SEQ ID NO:2. A nucleic acid molecule encoding an amino-terminal peptide fusion (6-His tag, thrombin cleavage site, S-tag, enterokinase, and Arabidopsis LTP minus the first 23 amino acid putative secretory leader sequence, in that order) is set forth in SEQ ID NO:3. The fusion polypeptide encoded by SEQ ID NO:3 is set forth in SEQ ID NO:4. An example of a homologous LTP is a cDNA encoding a Thellungiella halophia LTP set forth in SEQ ID NO:5 (Accession NO. AF499715). The Thellungiella halophia LTP polypeptide encoded by SEQ ID NO:5 is set forth in SEQ ID NO:6. Another example of a homologous LTP is a cDNA encoding a Brassica napus LTP set forth in SEQ ID NO:7 (Accession NO. AF101038). The Brassica napus LTP polypeptide encoded by SEQ ID NO:7 is set forth in SEQ ID NO:8.

In one embodiment, the LTP is an Arabidopsis LTP. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii.

In various embodiments, the LTP can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

LTP polypeptides having at least 40% sequence identity with Arabidopsis LTP (SEQ ID NO:2) protein are also useful in the methods of the invention. In one embodiment, the sequence identity is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence identity in ascending order with Arabidopsis LTP (SEQ ID NO:2) protein. In addition, it is preferred that LTP polypeptides of the invention have at least 10% of the activity of Arabidopsis LTP (SEQ ID NO:2) protein. LTP polypeptides of the invention have at least 10%, 15%, 20%, 25%, 30%, 35%, 40%, 45%, 50%, 55%, 60%, 65%, 70%, 75%, 80%, 85% or at least 90% of the activity of Arabidopsis LTP (SEQ ID NO:2) protein.

Polypeptides consisting essentially of SEQ ID NO:2 are also useful in the methods of the invention. For the purposes of the present invention, a polypeptide consisting essentially of SEQ ID NO:2 has at least 90% sequence identity with Arabidopsis LTP (SEQ ID NO:2) and at least 10% of the activity of SEQ ID NO:2. A polypeptide consisting essentially of SEQ ID NO:2 has at least 90%, 91%, 92%, 93%, 94%, 95%, 96%, 97%, 98%, or 99% sequence identity with SEQ ID NO:2 and at least 25%, 50%, 75%, or 90% of the activity of Arabidopsis LTP (SEQ ID NO:2). Examples of polypeptides consisting essentially of SEQ ID NO:2 include, but are not limited to, polypeptides having the amino acid sequence of SEQ ID NO:2 with the exception that one or more of the amino acids are substituted with structurally similar amino acids providing a “conservative amino acid substitution.” Conservative amino acid substitutions are well known to those of skill in the art. Particular examples of polypeptides consisting essentially of SEQ ID NO:2 include polypeptides having 1, 2, or 3 conservative amino acid substitutions relative to SEQ ID NO:2.

Other examples of polypeptides consisting essentially of SEQ ID NO:2 include polypeptides having the sequence of SEQ ID NO:2, but with truncations at either or both the 3′ and the 5′ end. For example, polypeptides consisting essentially of SEQ ID NO:2 include polypeptides having 1, 2, or 3 amino acids residues removed from either or both 3′ and 5′ ends relative to SEQ ID NO:3. Additional examples of polypeptides consisting essentially of SEQ ID NO:2 are LTP polypeptides in which the putative secretory leader sequence is absent, an example of which is the polypeptide of SEQ ID NO:4. In addition, LTP polypeptides consisting essentially of SEQ ID NO:2 can be fusion proteins, such as SEQ ID NO:4, in which a LTP polypeptide is fused with another polypeptide or amino acid sequence to aid in secretion and/or purification as is known to those of skill in the art.

Fragments of a LTP polypeptide are useful in the methods of the invention. In one embodiment, the LTP fragments include an intact or nearly intact epitope that occurs on a biologically active wild-type LTP. For example, the fragments comprise at least 10 consecutive amino acids of LTP of SEQ ID NO:2. The fragments comprise at least 15, 20, 25, 30, 35, 40, 50, 60, 70, 80, 90, 100 or at least 114 consecutive amino acid residues of LTP of SEQ ID NO:2. In addition, fragments of homologous LTP's are useful in the methods of the invention. Polypeptides comprising at least 50 amino acids having at least 50%, 60%, 70%, 80%, 90%, 95%, 96%, 97%, 98% or 99% sequence identity with at least 50 consecutive amino acid residues of SEQ ID NO:2 are useful in the methods of the invention. In one embodiment, the fragment is from an Arabidopsis LTP. In one embodiment, the fragment contains an amino acid sequence conserved among plant LTP's.

Thus, in another embodiment, the invention provides a method for identifying a compound as a candidate for a herbicide, comprising: contacting a compound with a LTP polypeptide selected from the group consisting of a LTP polypeptide set forth in SEQ ID NO:2 or SEQ ID NO:4; a LTP polypeptide consisting essentially of SEQ ID NO:2; a LTP polypeptide comprising at least 10 consecutive amino acids of SEQ ID NO:2; a LTP polypeptide having at least 50% sequence identity with SEQ ID NO:2; and a polypeptide comprising at least 50 amino acids having at least 50% sequence identity with at least 50 consecutive amino acid residues of SEQ ID NO:2; and detecting the presence and/or absence of binding between the compound and the polypeptide, wherein binding indicates that the compound is a candidate for a herbicide.

Any technique for detecting the binding of a ligand to its target is useful in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with an LTP protein or a homologue, fragment or variant thereof, the unbound protein is removed and the bound LTP is detected. In a preferred embodiment, bound LTP is detected using a labeled binding partner, such as a labeled antibody. In a variation of this assay, LTP is labeled prior to contacting the immobilized candidate ligands. Preferred labels include fluorescent or radioactive moieties. In other embodiments of the invention, detection methods include fluorescence correlation spectroscopy (FCS) and FCS-related confocal nanofluorimetric methods.

In another embodiment of the invention, compounds are tested as candidate herbicides based on ability to inhibit LTP enzyme activity. The compounds are tested using either in vitro or cell based enzyme assays. Alternatively, compounds are tested by direct application to a plant or plant cell, or expressing it therein, and monitoring the plant or plant cell for changes or decreases in growth, development, viability or alterations in gene expression.

A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

The ability of a compound to inhibit LTP activity can be detected using in vitro enzymatic assays in which the disappearance of a substrate or the appearance of a product is directly or indirectly detected. LTP catalyzes the transfer of lipid substrates. Methods for measuring the progression of a LTP enzymatic reaction and/or a change in the binding of a substrate lipid molecule, include spectrophotometry, fluorimetry, mass spectroscopy, thin layer chromatography (TLC) and reverse phase HPLC. In one embodiment of the invention, LTP enzyme activity is measured by detecting the binding of a fluorescently labeled substrate lipid molecule to a LTP polypeptide. With this method, the ability of a compound to inhibit the enzymatic activity of a LTP is measured by relative binding of the labeled substrate to the LTP in the presence and absence of the compound. A change in substrate binding to LTP in the presence of the compound indicates the compound as a herbicide candidate.

Thus, the invention provides a method for identifying a compound as a candidate herbicide, comprising: contacting a lipid substrate with a LTP polypeptide in the presence and absence of a compound; and determining a change in binding of the lipid substrate to the LTP polypeptide in the presence and absence of the compound, wherein a change in binding indicates that the compound is a candidate for a herbicide. In one embodiment of the invention, the LTP is the polypeptide set forth in SEQ ID NO:2. In another embodiment, the LTP is the polypeptide set forth in SEQ ID NO:4. In another embodiment, the LTP is a polypeptide consisting essentially of SEQ ID NO:2. In another embodiment, the LTP is an Arabidopsis LTP polypeptide. In another embodiment, the LTP is a plant LTP. In another embodiment the LTP is a homologous LTP. In another embodiment the LTP is Thellungiella halophia LTP set forth in SEQ ID NO:6. In another embodiment the LTP is Brassica napus LTP set forth in SEQ ID NO:8.

Enzymatically active fragments of Arabidopsis LTP set forth in SEQ ID NO:2 are also useful in the methods of the invention. For example, an enzymatically active polypeptide comprising at least 50 consecutive amino acid residues and at least 10% of the activity of Arabidopsis LTP set forth in SEQ ID NO:2 are useful in the methods of the invention. In addition, fragments of homologous LTP's are useful in the methods of the invention. Enzymatically active polypeptides having at least 10% of the activity of SEQ ID NO:2 and at least 50%, 60%, 70%, 80%, 90%, 95%, 96%, 97%, 98% or 99% sequence identity with at least 50 consecutive amino acid residues of SEQ ID NO:2 are useful in the methods of the invention. Most preferably, the enzymatically active polypeptide has at least 50% sequence identity with at least 50 consecutive amino acid residues of SEQ ID NO:2 and at least 25%, 75% or at least 90% of the activity thereof.

Thus, the invention provides a method for identifying a compound as a candidate herbicide, comprising: contacting a lipid substrate with a LTP polypeptide in the presence and absence of a compound, wherein the LTP polypeptide is selected from the group consisting of: a polypeptide consisting essentially of SEQ ID NO:2, a polypeptide having at least 50% sequence identity with Arabidopsis LTP set forth in SEQ ID NO:2 and having at least 10% of the activity thereof, a polypeptide comprising at least 50 consecutive amino acids of Arabidopsis LTP set forth in SEQ ID NO:3 and having at least 10% of the activity thereof, and a polypeptide comprising at least 50 amino acids, having at least 50% sequence identity with at least 50 consecutive amino acids of Arabidopsis LTP set forth in SEQ ID NO:2 and having at least 10% of the activity thereof; and determining a change in binding of the lipid substrate to the LTP polypeptide in the presence and absence of the compound, wherein a change in binding indicates that the compound is a candidate for a herbicide.

For the in vitro enzymatic assays, LTP protein and derivatives thereof may be purified from a plant or may be recombinantly produced in and purified from a plant, bacteria or eukaryotic cell culture. Preferably LTP proteins are produced using a baculovirus, E. coli or yeast expression system. Methods for purifying LTP are found, for example, in Dieryck et al. (1995) Protein Expr. Purif. 6:597-603 and in Lullien-Pellerin et al. (1999) Eur. J. Biochem. 260:861-868. Purification of the LTP polypeptide set forth in SEQ ID NO:4 is described herein in Example 6. Other methods for the purification of LTP proteins and polypeptides are known to those skilled in the art.

As an alternative to in vitro assays, the invention also provides plant based assays. In one embodiment, the invention provides a method for identifying a compound as a candidate for a herbicide, comprising: a) measuring the expression or activity of a LTP in a plant, or tissue thereof, in the absence of a compound; b) measuring the expression or activity of the LTP in the plant, or tissue thereof, in the presence of the compound; and c) comparing the expression or activity of the LTP in steps (a) and (b), wherein an altered expression or activity in the presence of the compound indicates that the compound is a candidate for a herbicide. In one embodiment, the plant or tissue thereof, is Arabidopsis.

In the methods of the invention, expression of a LTP in a plant, or tissue thereof, is measured by detecting the LTP primary transcript or mRNA, LTP polypeptide, or LTP enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting LTP RNA include, but are not limited to, amplification assays such as quantitative PCR, and/or hybridization assays such as Northern analysis, dot blots, slot blots, in-situ hybridization, transcriptional fusions using an LTP promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system is useful to detect LTP protein expression. For detection using gene reporter systems, a polynucleotide encoding a reporter protein is fused in frame with LTP, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2: 1104; Prost et al. (1986) Gene 45: 107-111), β-galactosidase (Nolan et al. (1988) Proc Natl Acad Sci USA 85: 2603-2607), alkaline phosphatase (Berger et al. (1988) Gene 66: 10), luciferase (De Wet et al. (1987) Mol Cell Biol 7: 725-737), β-glucuronidase (GUS), fluorescent proteins, chromogenic proteins and the like. Methods for detecting LTP activity are described above.

Chemicals, compounds, or compositions identified by the above methods as modulators of LTP expression or activity are useful for controlling plant growth. For example, compounds that inhibit plant growth are applied to a plant or expressed in a plant to prevent plant growth. Thus, the invention provides a method for inhibiting plant growth, comprising contacting a plant with a compound identified by the methods of the invention as having herbicidal activity.

Herbicides and herbicide candidates identified by the methods of the invention are useful for controlling the growth of undesired plants, including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants were grown in Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻²s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### Example 1

### Construction of a Transgenic Plant Expressing the Driver

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter. A driver expression cassette was introduced into Arabidopsis thaliana by agroinfection. Transgenic plants that stably express the driver transcription factor were obtained.

### Example 2

### Construction of LTP Antisense Expression Cassettes in a Binary Vector

A fragment of the Arabidopsis thaliana cDNA corresponding to SEQ ID NO:1 was ligated into the PacI/AscI sites of an E.coli/Agrobacterium binary vector in the antisense orientation to yield an antisense expression cassette and a constitutive chemical resistance expression cassette located between right and left T-DNA borders. In this construct, transcription of the antisense RNA is controlled by an artificial promoter active only in the presence of the driver transcription factor described above. The artificial promoter contains four contiguous binding sites for the GAL4 transcriptional activator upstream of a minimal promoter comprising a TATA box. The ligated DNA was transformed into E.coli. Kanamycin resistant clones were selected and purified. DNA was isolated from each clone and characterized by PCR and sequence analysis confirming the presence of the antisense expression cassette.

### Example 3

### Transformation of Agrobacterium with the LTP Antisense Expression Cassette

The binary vector described in Example 2 was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### Example 4

### Construction of Arabidopsis LTP Antisense Target Plants

The LTP antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis TI seedlings were selected. Approximately 70 mg seeds from an agrotransformed plant were mixed approximately 4:1 with sand and placed in a 2 ml screw cap cryo vial. One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. The herbicide resistant plants represented stably transformed T1 plants.

### Example 5

### Effect of LTP Antisense Expression in Arabidopsis Seedlings

The T1 LTP antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. Antisense expression of the LTP gene resulted in seedlings that displayed stunted growth and chlorosis, indicating that the LTP encoding gene is essential for normal plant growth and development.

### Example 6

### Cloning, Expression, and Purification of the LTP Protein

The following protocol was performed to obtain purified LTP polypeptide set forth in SEQ ID NO:4. A gene encoding LTP was cloned by RT-PCR from Arabidopsis thaliana. After removal of a 23 aa putative secretory leader sequence, the truncated form of the gene was ligated into an E. coli expression vector as follows. Total RNA was collected from 14-day-old Arabidopsis thaliana seedlings using published protocols, and reagents (Trizol) from Life Technologies, Inc. (Rockville, Md.). One μl of 10 μM custom oligo, AAT TGC GGC CGC TCA CTT GAT GTT GTT GCA (SEQ ID NO:9), was incubated with 1 μg of total RNA in a reverse transcriptase polymerase chain reaction (RT-PCR) (Invitrogen kit) according to the manufacturer's recommendations. The LTP cDNA was then selectively amplified by PCR with the primer pair ATT GGT ACC GCA ATC TCA TGT GGC ACA (SEQ ID NO:10) and AAT TGC GGC CGC TCA CTT GAT GTT GTT GCA (SEQ ID NO:11). The resulting PCR product, and plasmid pET30c(+) (Novagen, Madison, Wis.) were digested with restriction endonucleases KpnI and NotI as directed by the manufacturer (New England Biolabs, Beverly, Mass.). Ligation of these two linear DNAs into the resulting recombinant clone pET30c-LTP was accomplished by following instructions included with T4 DNA ligase (New England Biolabs). The integrity of the above clone comprising the sequence set forth in SEQ ID NO:3 was veri lied by DNA sequence analysis.

The Arabidopsis LTP N-terminal His-tag fusion protein (SEQ ID NO:4) encoded by pET30c-LTP expression vector, was expressed in E. coli and purified by affinity chromatography as follows. Pellets of E. coli culture expressing LTP fusion protein were resuspended in lysisbuffer: 50 mM sodium phosphate, pH 7.0, 300 mM NaCl, 1 mg/ml Lysozyme, 0.5 μl/ml Benzonase, 8 mM 2-mercaptoethanol, 150 μl protease inhibitor cocktail (Sigma, P-8849). The crude extract was incubated on ice for 1 h and sonicated for 5×20 sec. The homogenate was centrifuged at 17,500 g for 30 min. The supernatant was incubated for 30 min at 4° C. with Ni-NTA bead solution (Qiagen) and equilibrated with binding buffer (50 mM sodium phosphate, pH 7.0, 300 mM NaCl).

The beads were washed with 10 column volumes wash buffer (50 mM imidazole in binding buffer). Bound LTP protein was eluted with elution buffer (500 mM imidazole in binding buffer). LTP was desalted with NAP25 columns (Amersham Pharmacia). Protein was stored in 50 mM Tris/HCl pH 7.5 containing 10% glycerol at −80° C. Protein was analyzed using 4-20% SDS-PAGE, and transferred to 0.2 μm nitrocellulose membrane by the method of Towbin et al. (1979) Biotechnology 24:145-149. LTP was detected with anti-his primary antibody, alkaline phosphatase conjugated secondary antibody and BCIP/NBT reagent.

While the foregoing describes certain embodiments of the invention, those skilled in the art will understand that variations and modifications may still fall within the scope of the invention.

What is claimed is:

1. A method for identifying a compound as a candidate for a herbicide, comprising:
a) contacting a LTP polypeptide with a compound; and
b) detecting the presence or absence of binding between the compound and the LTP polypeptide, wherein binding indicates that the compound is a candidate for a herbicide.

2. The method of claim 1, wherein the LTP polypeptide is a plant LTP polypeptide.

3. The method of claim 1, wherein the LTP polypeptide is an Arabidopsis LTP polypeptide.

4. The method of claim 1, wherein the LTP polypeptide is SEQ ID NO:4.

5. A method for identifying a compound as a candidate for a herbicide, comprising:
a) contacting a compound with a polypeptide selected from the group consisting of:
i) a polypeptide consisting essentially of SEQ ID NO:2;
ii) a polypeptide having at least ten consecutive amino acids of SEQ ID NO:2;
iii) a polypeptide having at least 50% sequence identity with SEQ ID NO:2; and
iv) a polypeptide comprising at least 50 amino acids having at least 50% sequence identity with at least 50 consecutive amino acids of SEQ ID NO:2; and
b) detecting the presence and/or absence of binding between the compound and the polypeptide, wherein binding indicates that the compound is a candidate for a herbicide.

6. A method for identifying a compound as a candidate for a herbicide, comprising:
a) measuring the activity of a LTP in the presence and absence of a compound, wherein an alteration of the LTP activity in the presence of the compound indicates the compound as a candidate for a herbicide.

7. The method of claim 6, wherein the LTP is plant LTP.

8. The method of claim 7, wherein the plant is a dicot.

9. The method of claim 7, wherein the plant is a monocot.

10. The method of claim 7, wherein the plant is other than a C3 plant.

11. The method of claim 7, wherein the plant is other than a C4 plant.

12. The method of claim 6, wherein the LTP is an Arabidopsis LTP.

13. The method of claim 6, wherein the LTP is SEQ ID NO:4.

14. The method of claim 6, wherein the LTP is a LTP polypeptide consisting essentially of SEQ ID NO:2.

15. The method of claim 6, wherein the LTP is a LTP polypeptide selected from the group consisting of:
a) a polypeptide having at least 50% sequence identity with SEQ ID NO:2 and at least 10% of the activity of SEQ ID NO:2;
b) a polypeptide comprising at least 50 consecutive amino acids of SEQ ID NO:2 and having at least 10% of the activity of SEQ ID NO:2; and
c) a polypeptide comprising at least 50 amino acids having at least 50% sequence identity with at least 50 consecutive amino acids of SEQ ID NO:2 and having at least 10% of the activity of SEQ ID NO:2.

16. A method for identifying a compound as a candidate for a herbicide, comprising:
a) contacting a LTP polypeptide with a lipid substrate in the presence and absence of a compound; and
b) determining a change in binding of the lipid substrate in the presence and absence of the compound, wherein a change in the binding of the lipid substrate indicates that the compound is a candidate for a herbicide.

17. The method of claim 16, wherein the LTP is a plant LTP.

18. The method of claim 17, wherein the plant is a dicot.

19. The method of claim 17, wherein the plant is a monocot.

20. The method of claim 17, wherein the plant is other than a C3 plant.

21. The method of claim 17, wherein the plant is other than a C4 plant.

22. The method of claim 17, wherein the LTP is an Arabidopsis LTP.

23. The method of claim 17, wherein the LTP is SEQ ID NO:4.

24. The method of claim 16, wherein the LTP is a LTP polypeptide consisting essentially of SEQ ID NO:2.

25. The method of claim 16, wherein the LTP is a LTP polypeptide selected from the group consisting of:
a) a polypeptide having at least 50% sequence identity with SEQ ID NO:2 and at least 10% of the activity of SEQ ID NO:2;
b) a polypeptide comprising at least 50 consecutive amino acids of SEQ ID NO:2 and having at least 10% of the activity of SEQ ID NO:2; and
c) a polypeptide comprising at least 50 amino acids having at least 50% sequence identity with at least 50 consecutive amino acids of SEQ ID NO:2 and having at least 10% of the activity of SEQ ID NO:2.

26. A method for identifying a compound as a candidate for a herbicide, comprising:
a) measuring the expression of a LTP in a plant, or tissue thereof, in the presence and absence of a compound; and
a) comparing the expression of the LTP in the presence and absence of the compound, wherein an altered expression in the presence of the compound indicates that the compound is a candidate for a herbicide.

27. The method of claim 26, wherein the plant is Arabidopsis.

28. The method of claim 26, wherein the expression of the LTP is measured by detecting the LTP mRNA.

29. The method of claim 26, wherein the expression of the LTP is measured by detecting the LTP polypeptide.

30. The method of claim 26, wherein the expression of the LTP is measured by detecting the LTP polypeptide enzyme activity.

31. An isolated nucleic acid comprising a nucleotide sequence that encodes the polypeptide of SEQ ID NO:4.

32. An isolated nucleic acid comprising a nucleotide sequence that encodes a polypeptide consisting essentially of SEQ ID NO:2.

33. A recombinant polypeptide consisting essentially of the amino acid sequence of SEQ ID NO:2.

34. A recombinant polypeptide comprising the amino acid sequence of SEQ ID NO:4.
